(19) United States
(12) Patent Application Publication
(10) Pub. No.: US 2021/0298626 A1
(43) Pub. Date: Sep. 30, 2021

(54) METHOD AND SYSTEM FOR PROCESSING ECG DATA

(71) Applicant: GE Precision Healthcare LLC, Wauwatosa, WI (US)

(72) Inventors: Long Yu, Wauwatosa, WI (US); Brian J. Young, Wauwatosa, WI (US); Joel Qiuzhen Xue, Wauwatosa, WI (US); Gordan Ian Rowlandson, Wauwatosa, WI (US)

(73) Assignee: GE Precision Healthcare LLC, Wauwatosa, WI (US)

(21) Appl. No.: 16/834,785

(22) Filed: Mar. 30, 2020

(51) Int. Cl. A61B 5/04; A61B 5/044; A61B 5/0408; G16H 10/60; G06F 16/28; G16H 50/20

(52) U.S. Cl. CPC A61B 5/04012; A61B 5/044; G16H 50/20; G16H 10/60; G06F 16/285; A61B 5/04085

(57) ABSTRACT

A method of processing ECG data includes generating a first feature set with a trained neural network using ECG data and processing a patient's ECG data using a criteria-based algorithm to generate a second feature set. The patient's ECG data is then clustered into a number of clusters based on the first feature set and the second feature set to generate clustered ECG data. The clustered ECG data is presented to a user via a user interface, and user input is received from the user via the user interface regarding the clustered ECG data. A feature vector is defined based on the user input and the feature vector is applied to at least a portion of the patient's ECG data to generate revised clustered ECG data. The revised clustered ECG data is then presented to the user via the user interface.

### BACKGROUND

This disclosure generally relates to systems and methods for processing ECG data, and more particularly to systems and methods providing human-guided machine learning for efficient editing of ECG data.

An electrocardiogram (ECG) is often used to diagnose arrhythmias and other cardiac abnormalities. As some cardiac abnormalities are intermittent, they cannot be readily captured in a standard 12-lead resting ECG, and thus many patients are monitored using Holter monitors or other ambulatory ECG monitors to continuously or intermittently record ECG data from a patient over an extended period of time. Systems and methods for interpreting electrocardiograph (ECG) waveforms are currently available to assist a clinician in interpreting waveforms and assessing patient cardiac health based on ECG waveforms. Currently available systems and methods generally process ECG waveform data and provide suggested interpretations based thereon. These currently available systems and methods generally require processing ECG waveforms to identify certain predefined waveform features, and those identified features provide the basis for arrhythmia detection. For example, many interpretation systems utilize proprietary feature extraction algorithms.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one embodiment, a method of processing ECG data includes generating a first feature set with a trained neural network using ECG data and processing a patient's ECG data using a criteria-based algorithm to generate a second feature set. The patient's ECG data is then clustered into a number of clusters based on the first feature set and the second feature set to generate clustered ECG data. The clustered ECG data is presented to a user via a user interface, and user input is received from the user via the user interface regarding the clustered ECG data. A feature vector is defined based on the user input and the feature vector is applied to at least a portion of the patient's ECG data to generate revised clustered ECG data. The revised clustered ECG data is then presented to the user via the user interface.

One embodiment of the system for processing ECG data includes a trained neural network configured to generate a first feature set based on ECG data and a criteria-based feature module configured to process a patient's ECG data using a criteria-based algorithm to generate a second featured set. A user-guided clustering module is configured to cluster the patient's ECG data into a number of clusters based on the first feature set and the second feature set to generate clustered ECG data and then present the clustered ECG data to a user. User input is received from the user regarding the clustered ECG data and a feature vector is defined based on the user input. The feature vector is applied to at least a portion of the patient's ECG data to generate revised clustered ECG data.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 depicts an exemplary system for processing ECG data in accordance with one embodiment of the present disclosure;

FIG. 2 depicts another exemplary system for processing ECG data, and Holter data in particular, in accordance with another embodiment of the present disclosure;

FIGS. 3-4 depict exemplary methods of processing ECG data in accordance with the present disclosure;

FIG. 5 depicts an exemplary user interface and method for processing ECG data in accordance with the present disclosure.

### DETAILED DESCRIPTION

The inventors have recognized that editing ECG data, and particularly ambulatory ECG data that is continuously recorded, is time-consuming and labor-intensive. ECG data, and particularly ambulatory and/or continuously recorded ECG data, tends to be artifact laden and needs to be edited and pre-processed prior to diagnostic review by a physician. Editing Holter ECG data, for example, involves repetitive edits by a technician or other trained professional in order to remove noise and accurately categorize and process the ECG data recorded from Holter monitors. While highly laborious and repetitive for any single recording of a given patient's ECG, no automated system or strategy has yet been developed to accurately automate the editing process for Holter data or other ambulatory ECG data. Editing strategies are intuitive, heuristic, and subjective, and thus edits vary among technicians for a given recording and is heavily influenced by the type of Holter system on which they were trained, their level of experience, and their level of understanding of electrocardiography. Moreover, the range of editing strategies will vary among different recordings and different patients because of the wide range of both physiologic and noise content. Thus, the ECG editing process cannot be easily generalized for purposes of developing a completely automated and deterministic ECG processing algorithm, whether it be a criteria-based algorithm or a fully pre-trained machine learning algorithm. Thus, existing ECG processing systems and methods all utilize human editing as part of the ECG processing strategy. That human editing is repetitive and laborious for a given ECG recording, where the human may need to repeat the same task over and over, such as to remove a particular artifact or identify a waveform feature over time in a patient's ECG.

In view of their recognition of the foregoing challenges and problems in the relevant art, the inventors have developed the disclosed system and method that encodes human ECG editing actions using a fast machine learning algorithm and reproduces that same editing strategy across a patient's ECG dataset (or some portion thereof) in order to avoid repetitive human input. Thus, the system is configured to receive initial editing input from a human user, and thus to capture the intuitive and subjective judgment of the trained user, and then to automatically repeat that action throughout an ECG recording for a patient. Thereby, the disclosed system and method remove the laboriousness and time-consuming portions of the human editing process but capture the intuition and skill of the trained human technician. Thereby, the efficiency and consistency of ECG editing, and ambulatory ECG editing in particular, are vastly increased by providing a cost-effective solution that focuses on reducing human interaction in the editing process, but not eliminating it.

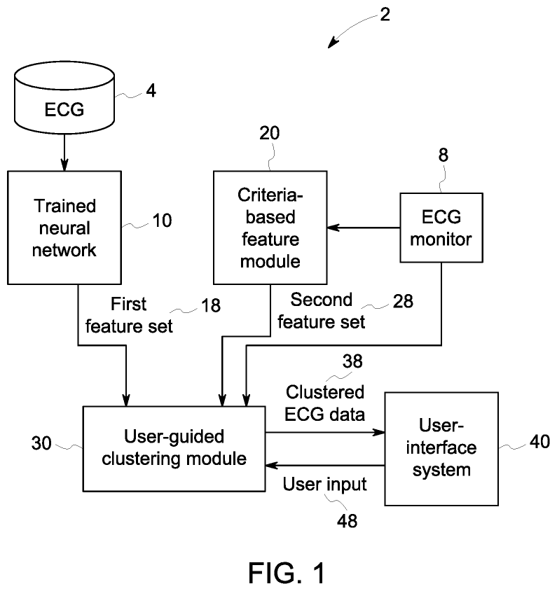

FIG. 1 depicts one embodiment of the present disclosure which comprises two pre-processing portions and a user-guided module that receives editing input from the user via a user interface, formulates abstractions based on those edits, and revises the ECG data processing accordingly. In one embodiment, the system includes a trained neural network 10 configured to generate a first feature set based on ECG data from an ECG database 4. The ECG database 4 contains ECG data from multiple patients, such as a large ECG database providing thousands or hundreds of thousands of ECG recordings from a multitude of patients. The ECG database implemented by the MUSE Cardiology Information System by General Electric Company of Synecdoche, New York is one example. The pre-trained neural network 10 is trained on large amounts of available ECG data to generate a first feature set 18 to extract features from the ECG data that are lead-independent and are generalized across the available ECG data utilized for training. Thus, the first feature set 18 generated by the trained neural network 10, once generated, is static and patient-independent. To provide just one example, the trained neural network 10 may generate 32 features, which are lead-independent. Once the 32 features are generated, those features are static and utilized to process all patient ECG data.

The pre-processing portion also includes a criteria-based feature module 20 that utilizes a criteria-based algorithm to generate a second feature set 28 based on raw ECG data recorded for a patient by an ECG monitor 8. The criteria-based algorithms may include existing beat analyzer algorithms that analyze rhythm and morphology, for example, shape, amplitude, and timing, of waveforms. For instance, the criteria-based algorithm may include at least one waveform-shape categorization algorithm specialized for processing Holter ECG data or other ambulatory ECG data. Alternatively or additionally, the criteria-based algorithms may include the Marquette 12SL ECG processing algorithms by General Electric Company. Alternatively or additionally, the criteria-based algorithm may include Holter processing algorithms for processing ambulatory ECG data acquired by Holter acquisition devices. Thus, the second feature set 28 is generated based on the patient's ECG data and thus contains, at least in part, features that are generated based on the patient's data and are specific thereto.

The first feature set 18 generated by the trained neural network 10 and the second feature set 28 generated by the criteria-based feature module 20, as well as the patient's raw ECG data from the ECG monitor 8, are provided to a user-guided clustering module 30 that is configured to facilitate human-guided editing automation of the patients ECG data. The user-guided clustering module 30 is configured to cluster the patient's ECG data and present initial clustered ECG data 38 to a user, which is generally a trained ECG technician and/or a physician. The user provides user input to edit the clustered ECG data 38 or other classification information generated based thereon.

The user provides the user input 48, such as inputs editing the initial clustered ECG data 38, via a user interface system 40 configured to facilitate presentation of the clustered ECG data to a user and to receive user inputs. For example, the user interface system 40 may include standard ECG editing terminals comprising digital computer monitors for visually presenting the clustered ECG data to a user and one or more user input devices, such as a keyboard, mouse, touch screen, and/or any other existing user input device providing an appropriate means by which a user can provide input for ECG editing.

The user input 48, such as an edit to the initially-presented clustered ECG data, is generalized by defining a feature vector based on the user input 48. The user input may be any of various types of information inputted by user during the ECG editing process, such as an amplitude and/or a timing threshold to be utilized for grouping some or all of the ECG data, a label for one or more clusters of ECG data (e.g. “normal” or “abnormal”), or other types of user input provided during the editing process, including user edits which are being made using existing ambulatory ECG editing software.

A machine learning algorithm is utilized to extract a feature vector that characterizes the user input 48, such as an editing input provided by the user. For example, the machine learning algorithm may implement regularized linear inversion for processing the feature sets of the ECG data relevant to the user input 48. Examples are described below. The feature vector is then applied to at least a portion of the ECG data to generate revised clustered ECG data. For example, the user-guided clustering module 30 may be configured to re-cluster some or all of the patient's ECG dataset based on the feature vector, which may be used as an alternative or in addition to some or all of the features in the first feature set 18 and the second feature set 28. In various examples, the re-clustering may be performed in response to certain user input types and/or in response to a user input instructing re-clustering based on a proceeding editing input to the clustered ECG data. The revised cluster ECG data is then presented to the user, such as for further editing.

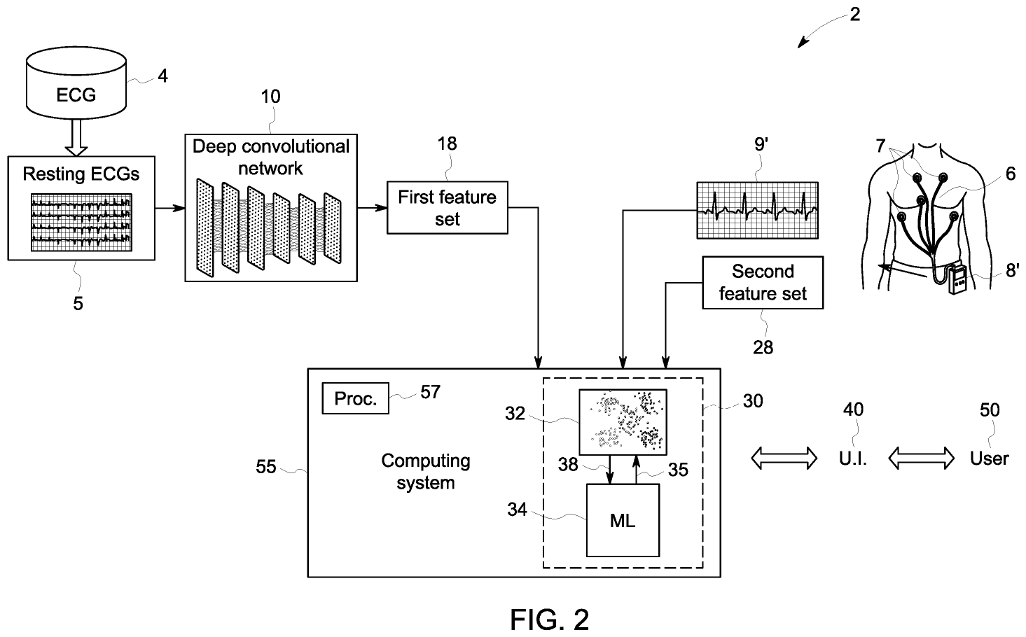

FIG. 2 depicts an embodiment of a system 2 for processing ECG data according to the present disclosure that is particularly configured for processing Holter ECG data 9′ recorded by a Holter monitor 8′. As is known to a person of ordinary skill in the art, a Holter monitor is a small, wearable ECG recording device that is worn by a patient for an extended period of time to continuously or periodically record ECG data from the patient. The Holter monitor 8′ receives ECG potentials recorded by five electrodes 7 attached to the patient 6, which are worn by the patient for an extended period of time while the patient conducts his or her normal activity. Alternatively, the Holter monitor may receive data from an electrode set that contains more or less than 5 electrodes. Thus, Holter monitors generate large amounts of ECG data, which may be stored internally within the Holter monitor. Alternatively, the Holter monitor may transmit the ECG data in part or entirety to another device or system for processing and review, in which case the entire system may be considered the Holter monitor. This ECG data is ambulatory ECG data 9′, which tends to contain a lot of artifacts. The cardiac signal needs to be isolated from the artifact, and furthermore, the massive amounts of ECG data need to be processed so that they are analyzable by a clinician, such as a physician.

Criteria-based ECG processing algorithms and software have been developed and utilized for many years that pre-process the ECG data to isolate and categorize the cardiac waveforms based on waveform shape, amplitude, and/or timing. For example, existing software may detect individual beats within the ECG data and categorize beats based on the morphology, amplitude, or timing of the waveform features. These detected values and qualities are captured in the second feature set 28 generated based on the patient's Holter ECG data 9′. For example, the criteria-based feature module 20 for generating the second feature set 28 may be contained and executed within the Holter monitor 8′, and thus generated as part of the data output from the Hotler monitor 8′. Alternatively, the criteria-based featured module 20, or at least portions thereof, may be contained and executed on an intermediary computing system or network that executes initial processing of the raw Holter ECG data 9′ prior to processing by the user-guided clustering module 30.

In the depicted example, the trained neural network 10 is a deep convolutional neural network (CNN) trained on ECG recordings of multiple patients stored in an ECG database 4. In the depicted example, the training data for the CNN 10 include resting ECGs 5, such as standard 12-lead ECG resting recordings. Standard 12-lead ECG resting recordings, such as ten second ECG recordings, are typically made in isolated clinical settings which are optimized for recording clean ECGs using well-placed electrodes on a patient that is lying still. Resting ECG recordings typically include little or no artifact and the diagnostically important waveform features are typically readily visible. Since the 12-lead resting ECG is the most common ECG test performed and has been performed for many decades, there is a large amount of such data to be used as training data for the CNN 10. The inventors have realized, that in certain embodiments, it may be beneficial to utilize resting ECG data 5 for training the neural network 10, even where the system 2 is configured for processing ambulatory ECG data. Mainly, where there is a lack of other types of training data, the inventors have recognized that it may be beneficial to train the neural network 10 using resting representative patterns formed from an aggregate of raw ECG complex. In certain embodiments, the resting representative patterns ECG data may be adapted with randomization, such as to introduce noise, waveform reverse, etc. so as to provide sufficiently diverse training data.

The first feature set 18 and the second feature set 28 are provided to a computing system 55 configured to facilitate and execute the user-guided editing processes described herein. The computing system 55 includes a processing system 57 and a storage system storing the user-guided clustering module 30 executable by the processing system 57 in order to perform the functions described herein. The computing system 55 may, in some embodiments, be implemented as a network, such as a cloud-implemented system.

The user-guided clustering module implements a clustering algorithm 32 to cluster the raw Holter ECG data 9′ into a number of clusters based on the first feature set 18 and the second feature set 28. For example, the clustering algorithm may be a k-means clustering algorithm for vector quantization of the patient's raw ECG data into “k” number of clusters based on “n” number of features, where each feature belongs to the cluster with the nearest mean. In certain embodiments, the clustering algorithm 32 may be configured to generate a predetermined number of clusters generally appropriate for ECG data. For example, a human heart is physiologically only capable of generating a certain number of waveform shapes, and thus the predetermined number of clusters may be defined based on the maximum number of shapes, or morphologies, that a human heart is capable of generating. To provide just one example, the clustering module 30 may be configured to cluster the patient's ECG data into ten clusters. One or more additional classes may be included to reject irregularly large amplitude or other common artifact waveforms that are not within a value range of the actual cardiac signal. In other embodiments, the clustering algorithm 32 may be configured to define the number of clusters based on the feature sets 18, 28 and/or the patient's raw ECG data. Other relevant constraints or starting conditions may include morphology-based assessments, such as whether the waveform meets criterion for defining normal, abnormal, and noisy waveforms. Alternatively or additionally, various constraints or starting conditions may be defined to accommodate patient position or activity, such as an initial number of clusters formed by the clustering module 30, etc. In still other embodiments, the user 50 may provide input to dictate how many clusters, or classes, to be generated by the k-means clustering algorithm 32.

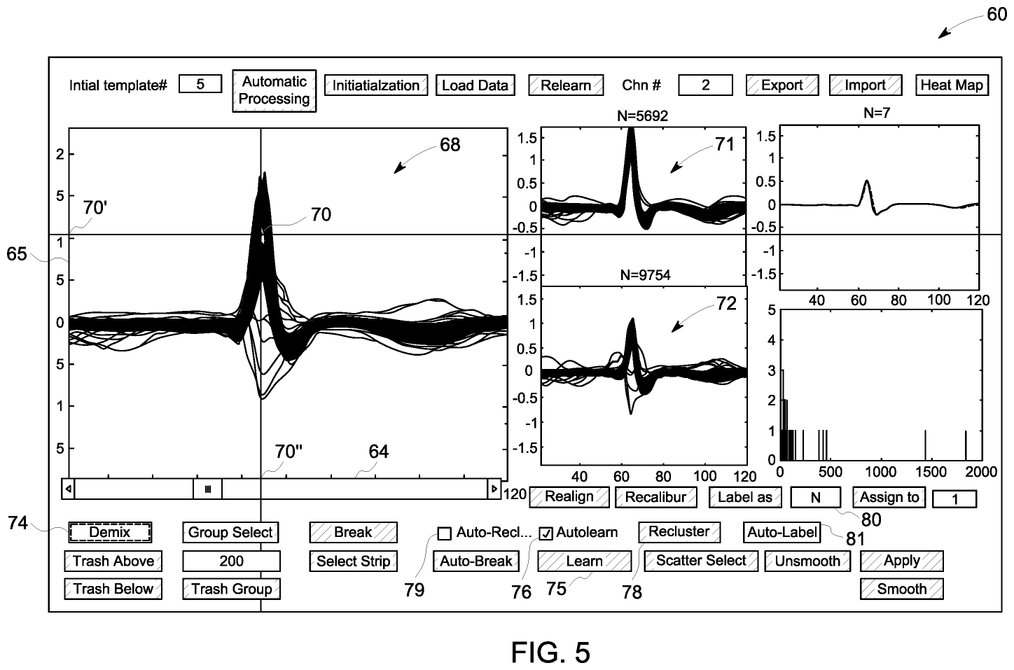

The initial clustered ECG data generated by the clustering algorithm 32 in the first instance is presented to the user 50 via the user interface system 40, which as described above can be any means by which an ECG technician or other trained user 50 can view and interact with processed ECG data, such as clustered ECG data 38. An exemplary user interface display 60 of portions of clustered ECG data 38 are presented at FIG. 5. In the example, one exemplary cluster 68 of ECG waveforms is graphically depicted, where all of the ECG beat waveforms grouped in the cluster 68 are overlaid with respect to a time axis 64 and a voltage axis 75.

The user 50 provides input regarding the clustered ECG data 38, such as input to edit the clustered ECG data presented on the user interface display 60 provided by the user interface system 40 or to classify or label the clustered ECG data 38. For example, the user input 48 may indicate one or more of an amplitude threshold, a timing threshold, and a label for the ECG data in one or more of the clusters. Exemplary user inputs are further described below with respect to FIG. 5.

The user-guided clustering module 30 is configured to process the user input and make abstractions based thereon, and to define a feature vector that captures the edit or other information inputted by the user so that the user input can be repeated across the entire ECG dataset for the patient. A feature is an individual measurable property or characteristic of the ECG data. A feature vector describes a set of features. The feature vector generated based on the user input contains information describing the important characteristics of the user input, which are identified by the machine learning algorithm 34 based on the user input and the feature sets of the relevant clustered ECG data to which the user-inputted edit pertains. For example, the machine learning algorithm 34 may be a regularized linear inversion model which minimizes the least squares objective function in order to approximate the user input. The features can take many forms and thus the information contained in the feature vector 35 may vary greatly based on the type of information provided by the user.

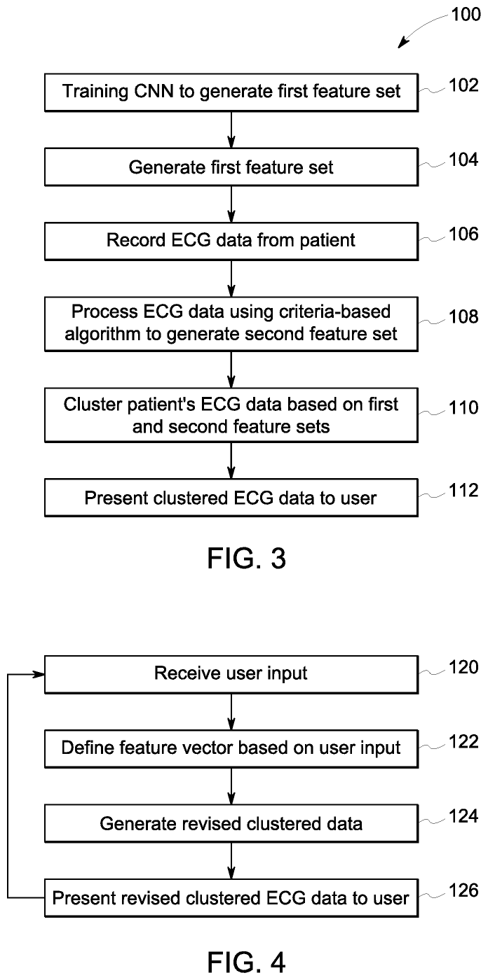

FIGS. 3 and 4 depict embodiments of methods 100 of processing ECG data in accordance with the present disclosure. FIG. 3 depicts method steps for preprocessing ECG data and generating initial cluster ECG data based on a patient's ECG data. FIG. 4 depicts steps for revising and editing the clustered ECG data using the machine learning algorithm 34 to learn based on the user input and apply the learned edit across the entire ECG dataset.

In FIG. 3, the method 100 includes first training a neural network at step 102 to generate the first feature set based on ECG data, such as a convolutional neural network (CNN) trained on a large database of ECG data from multiple patients. The first feature set generated at step 104 is then stored and used for ECG processing tasks for all patients. ECG data is recorded from a patient at step 106, such as from a Holter monitor or other ambulatory ECG monitor. The patient's ECG data is then processed at step 108 using a criteria-based algorithm or set of criteria-based algorithms, to generate a second feature set. The first and second feature sets and the patient's raw ECG data are then presented to a clustering module which clusters the patient's ECG data at step 110 based on the first and second feature sets. This initial clustered ECG data is then presented to the user at step 112.

User input is received at step 120, such as to modify the clustering or to label one or more of the clusters as shown at FIG. 4 and further exemplified at FIG. 5. Once a user input is provided that is to be learned, a feature vector is defined at step 122 based on the user input. The feature vector is then applied across at least a portion of the patient's ECG data to generate revised clustered ECG data at step 124. Depending on the feature vector and/or user instruction, application of the feature vector to the patient's ECG data may be different. Generating the revised clustered ECG data may include, for example, labeling all clusters based on a labeling input provided by a user. As another example, generating the revised clusters may include re-clustering some or all of the raw ECG data based on the feature vector generated based on user input providing revised clustering criteria. The revised clustered ECG data is then presented to the user at step 126.

FIG. 5 depicts an exemplary user interface display 60 and user interactions that may be learned by the system and automatically applied across the entire ECG dataset for the patient, or to a portion of the ECG dataset. A demixing example is illustrated where a threshold 70 is inputted by a user via the user interface, and a demix input 74 user input provided instructing division of the depicted cluster 68 into two sub-clusters 71 and 72. In the depicted example, the user identifies a threshold 70 by placing a cursor on a location of the graphed beat waveforms in the selected cluster 68. Here, the threshold 70 is an amplitude threshold at an identified time where all beat waveforms having an amplitude greater than the amplitude threshold 70′ at the identified time 70″ is divided into a first sub-cluster 71, and all beat waveforms having an amplitude less than the amplitude threshold 70′ at the time 70″ are divided into a second sub-cluster 72. In other embodiments, the threshold 70 may comprise a timing threshold at a particular amplitude, and the beat waveforms in the particular cluster divided into sub-clusters accordingly.

Where the user has instructed that the system learn based on the user-inputted threshold 70, then a feature vector is defined based on the at least two sub-clusters 71 and 72 of ECG data. Namely, the feature space X defining the cluster 68 is divided into feature space X1 and feature space X2. This action can then be encoded using a machine learning algorithm, such as a regularized linear inversion machine learning algorithm. In such an embodiment, a feature vector may be generated according to the following:

w=argmin(|WX−X1|²+|WX−X2|²)

Namely, the argument of the minimum is taken based on the difference between the group of weights in feature space X and the weights in feature spaces for each of the first sub-cluster 71 and the second sub-cluster 72. Thus, the feature vector W identifies the features used to demix, or split, the cluster 68 and reweights the feature space accordingly. That feature vector thus characterizes the demixing instruction provided by the user in a way that can be applied to some or all of the other clusters in the clustered ECG data 38 for the patient. Alternatively, a regularization, or optimization, factor may be utilized in order to learn using minimal samples. In such an embodiment, the action may be encoded and feature vector generated as:

w=argmin(|WX−X1|²+|WX−X2|²+λI)

where λ is a regularization factor and I is an identical matrix.

Once the feature vector is defined based on the at least two sub-clusters of ECG data, the feature vector can be utilized, such as to recluster some or all of the ECG data. User interface display 60 may provide user inputs to control the application of the feature vector, as well as to control generation of the feature vector. For example, the feature vector may be defined in order to learn the user-inputted modification based on user instruction via a “learn” instruction input 75 or a user instruction to put the system in “Auto-Learn” mode, such as represented at dial box 76. Namely, in Auto-Learn mode the system may automatically learn based on user inputs. Alternatively, the user may click the “learn” button 75 following an editing input, or series of editing inputs, so as to instruct the system to generate a feature vector based thereon. Similarly, the system may be configured to receive user input instructing reclustering based on a generated feature vector. In the example, a recluster button 78 enables a user to provide an input instructing that the patient's ECG data be reclustered based on the user input in order to generate revised clustered ECG data. Additionally, an “Auto-Recluster” mode may be available, which can be activated via dialbox 79, wherein the patient's ECG data is automatically reclustered upon generation of the feature vector.

The user input display 60 is also configured to enable user input to label the ECG data in one or more of the clusters. In the example, labeling user input can be provided at dial box 80, such as to label the ECG data in cluster 68 (and/or clusters 71 and 72) as normal “N” or abnormal “A”. In certain embodiments, other labeling inputs may be available, such as diagnostic labels indicating one or more diagnoses associated with, or based on, the ECG beat waveforms in the relevant cluster. The inputted label is then associated with the waveforms in the cluster 68, and/or the sub-clusters 71 and 72, and a feature vector is generated characterizing the labeling input based on the feature space X of the relevant clustered data. In certain embodiments, the user interface display 60 may be configured to facilitate user input to automatically apply the feature vector for the user-inputted label across all clusters, which in the depicted example is the “Auto-Label” button 81. Each of the clusters in the clustered ECG data 38 (or the reclustered ECG data if applicable) will be analyzed based on the feature vector for the user-inputted label and assessed as to whether the cluster should also be labeled as normal.

This written description uses examples to disclose the invention, including the best mode, and also to enable any person skilled in the art to make and use the invention. Certain terms have been used for brevity, clarity and understanding. No unnecessary limitations are to be inferred therefrom beyond the requirement of the prior art because such terms are used for descriptive purposes only and are intended to be broadly construed. The patentable scope of the invention is defined by the claims, and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

We claim:

1. A method of processing ECG data comprising:
generating a first feature set with a trained neural network using ECG data;
processing a patient's ECG data using a criteria-based algorithm to generate a second feature set;
clustering the patient's ECG data into a number of clusters based on the first feature set and the second feature set to generate clustered ECG data;
presenting the clustered ECG data to a user via a user interface;
receiving a user input via the user interface regarding the clustered ECG data;
defining a feature vector based on the user input;
applying the feature vector to at least a portion of the patient's ECG data to generate revised clustered ECG data; and
presenting the revised clustered ECG data to the user via the user interface.

2. The method of claim 1, wherein the feature vector is defined using a regularized linear inversion machine learning algorithm.

3. The method of claim 1, wherein the patient's ECG data is ambulatory ECG data.

4. The method of claim 3, wherein the criteria-based algorithm includes at least one algorithm for processing ECG data from a Holter monitor.

5. The method of claim 4, wherein the criteria-based algorithm includes at least one waveform shape categorization algorithm for processing the ECG data from the Holter monitor.

6. The method of claim 3, wherein the trained neural network is a convolutional neural network trained using resting ECG data of multiple patients.

7. The method of claim 1, wherein the number of clusters is a predetermined number of clusters.

8. The method of claim 1, wherein clustering the patient's ECG data into the number of clusters based on the first feature set and the second feature set includes clustering based on features describing waveform shape, waveform amplitude, and/or waveform timing.

9. The method of claim 1, wherein the user input indicates at least one of an amplitude threshold and a timing threshold.

10. The method of claim 9, further comprising dividing the ECG data in at least one of the number of clusters into at least two sub-clusters based on the amplitude threshold and/or the timing threshold, wherein the feature vector is defined based on features of each of the first feature set and the second feature set associated with the at least two sub-clusters of ECG data.

11. The method of claim 10, wherein the feature vector is defined based on a comparison of the features associated with ECG data in the two sub-clusters.

12. The method of claim 10, wherein the feature vector is defined based on the features associated with the two sub-clusters using a regularized linear inversion machine learning algorithm.

13. The method of claim 10, wherein generating revised clustered data includes reclustering at least a portion of the patient's ECG data based on the feature vector.

14. The method of claim 1, wherein the user input indicates a label for the ECG data in at least one of the number of clusters.

15. A system for processing ECG data, the system comprising:
a trained neural network configured to generate a first feature set based on ECG data;
a criteria-based feature module configured to process a patient's ECG data using a criteria-based algorithm to generate a second feature set;
a user-guided clustering module configured to:
cluster the patient's ECG data into a number of clusters based on the first feature set and the second feature set to generate clustered ECG data;
present the clustered ECG data to a user;
receive a user input from the user regarding the clustered ECG data;
define a feature vector based on the user input; and
apply the feature vector to at least a portion of the patient's ECG data to generate revised clustered ECG data.

16. The system of claim 15, wherein user-guided clustering module is configured to define the feature vector using a regularized linear inversion machine learning algorithm.

17. The system of claim 15, wherein the number of clusters is a predetermined number of clusters.

18. The system of claim 15, wherein user-guided clustering module is configured to perform k-means clustering of the patient's ECG data into the number of clusters based on the first feature set and the second feature set.

19. The system of claim 15, wherein the user input indicates at least one of an amplitude threshold, a timing threshold, and a label for the patient's ECG data in at least one of the number of clusters.

20. The system of claim 15, wherein the patient's ECG data is ambulatory ECG data and wherein the trained neural network is a convolutional neural network trained using an ECG database containing resting ECG data of multiple patients.
